(19) United States
(12) Patent Application Publication
(10) Pub. No.: US 2025/0001074 A1
(43) Pub. Date: Jan. 2, 2025

(54) MEMS MICROPUMP WITH SENSOR INTEGRATION TO DETECT ABNORMAL FUNCTION

(71) Applicant: AITA BIO INC., New York, NY (US)

(72) Inventors: Dilan Casanovas Mack, New York, NY (US); Jacob Menture, New York, NY (US)

(21) Appl. No.: 18/576,529

(22) PCT Filed: Aug. 3, 2022
(86) PCT No.: PCT/US22/39247

§ 371 Date: Jan. 4, 2024

Related U.S. Application Data
(60) Provisional application No. 63/229,115, filed on Aug. 4, 2021.

(51) Int. Cl. A61M 5/168; A61M 5/142

(52) U.S. Cl. CPC A61M 5/16854; A61M 5/14224; A61M 5/16881; A61M 2205/0244; A61M 2205/0294; A61M 2205/3334

(57) ABSTRACT

A MEMS device is disclosed that is configured as a micropump with an inlet port to receive fluid and an outlet port to release the fluid from the micropump. The MEMS device comprises first and second wafers, the first wafer configured as a membrane; a chamber defined by the first and second wafers for receiving fluid, the chamber configured to communicate with the inlet and outlet ports and defining a fluidic pathway between the inlet and outlet ports, wherein the first wafer is configured to deform creating a pressure difference within the chamber and thereby move fluid into or from the chamber via inlet and outlet ports, respectively; and first sensor and second sensors, in proximity to the inlet and outlet ports respectively, for sensing flow or pressure.

### CROSS-REFERENCE TO RELATED APPLICATIONS

This application claims priority to U.S. provisional application No. 63/229,115, filed on Aug. 4, 2021 entitled “MEMS MICROPUMP WITH SENSOR INTEGRATION”, which is incorporated by reference herein.

### FIELD OF INVENTION

The present invention relates to a MEMS micropump with sensor integration to detect abnormal function in the micropump or a medicament delivery device in which the micropump is a component.

### BACKGROUND OF THE INVENTION

Insulin pumps can help people with diabetes conveniently manage their blood sugar. These small devices deliver doses of insulin at specific times as a small continuous insulin does (basal insulin) or surges of insulin near mealtime (bolus insulin). Insulin patch pumps (sometimes referred to as “pods”) are typically affixed to a user's skin by an adhesive patch and the communicates wirelessly with a handheld controller as known to those skilled in the art. The patch pumps incorporate a micropump to deliver insulin through a cannula under a user's skin.

The life of a user (patient) depends on the proper functioning of these pumps. Abnormal pump function may create a potentially dangerous user event. Typical abnormal function results in under-infusion or over-infusion of insulin. Examples of abnormal pump function includes leaks, occlusions and air-bubble presence in the pumping line. Occlusion detection, for example, is important as occlusion may occur in the fluid pathway in numerous circumstances. Any undetected occlusion may result in an under-delivery of insulin because it remains undetected for a long period of time.

### SUMMARY OF THE INVENTION

A MEMS device configured as a micropump with sensor integration to detect abnormal function is disclosed. The detected abnormal function may occur within the micropump itself or an insulin delivery device in which the micropump is a component. The sensor integration includes sensors in proximity to the inlet and outlet ports of a fluidic pathway of the MEMS micropump.

In accordance with an example of this disclosure, a MEMS micropump comprising: a pump section including a chamber that is configured to increase and decrease in volume as fluid is received and released from the chamber respectively and an actuator for changing the volume within the chamber; a first valve section including an inlet port communicating with the chamber to receive the fluid; a second valve section including an outlet port communicating with the chamber to release the fluid from the chamber; first and second wafers configured to define the chamber and enable it to communicate with the inlet port and outlet port and define a fluid pathway between the inlet port, chamber and outlet port, wherein the first wafer is configured as a membrane that deforms in response to actuation of the actuator creating a pressure difference within the chamber as it increases and decreases in volume, thereby moving fluid into or out of the chamber via inlet and outlet ports, respectively; first sensor and second sensors, in proximity to the inlet and outlet ports respectively, for detecting abnormalities in fluid flow within the fluid pathway; and a third wafer joined to the second wafer configured to integrate the first sensor and second sensors into the inlet port and the outlet port.

In accordance with another example of this disclosure, a MEMS device is disclosed that is configured as a micropump with an inlet port to receive fluid and an outlet port to release the fluid from the micropump. The MEMS device comprises first and second wafers, the first wafer configured as a membrane; a chamber defined by the first and second wafers for receiving fluid, the chamber configured to communicate with the inlet and outlet ports and defining a fluidic pathway between the inlet and outlet ports, wherein the first wafer is configured to deform creating a pressure difference within the chamber and thereby move fluid into or from the chamber via inlet and outlet ports, respectively; and first sensor and second sensors, in proximity to the inlet and outlet ports respectively, for sensing flow or pressure.

### BRIEF DESCRIPTION OF DRAWINGS

FIG. 1 depicts a block diagram of an example MEMS micropump with sensor integration.

FIGS. 2-7 depict several stages-steps of the MEMS micropump fabrication and corresponding structure.

### DETAILED DESCRIPTION OF THE INVENTION

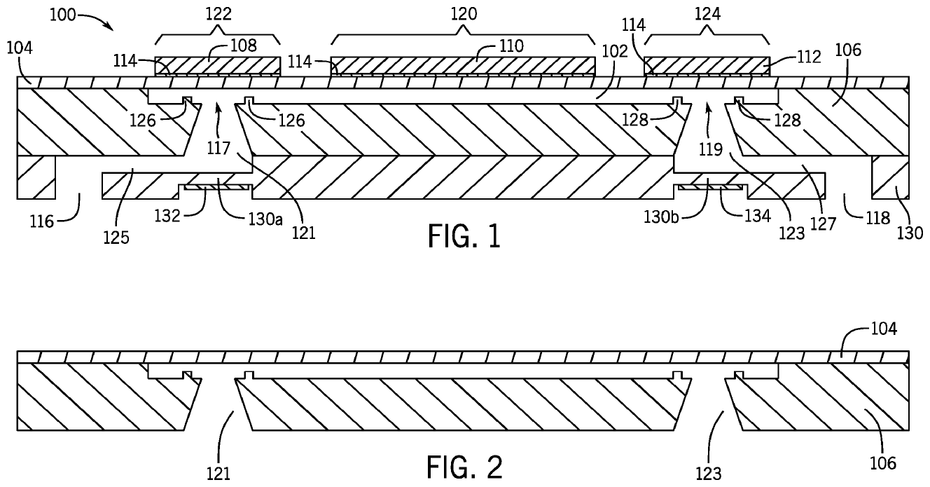

FIG. 1 depicts a cross sectional view of an example micropump or pump 100. Micropump 100 is part or a component of a device for delivering insulin (“delivering device” or “delivery device”) such as a patch pump, pumping unit or pump. The delivering device is part of an insulin infusion system for infusing insulin to a user (patient) for diabetes management (e.g., type 1). The pump incorporates several components including a reservoir, microcontroller unit (MCU), battery, insulin delivery needle, glucose monitor components (e.g., CGM sensor and needle) to name a few.

Micropump 100 is described herein as used for pumping insulin. However, micropump 100 (as part of delivering device) may however be used for other medicaments such as small molecule pharmaceutical solutions, large molecule or protein drug solutions, saline solutions, blood or other fluids known to those skilled in the art.

Micropump 100 is a MEMS (micro-electro-mechanical systems) device, as known to those skilled in the art, that can be used for pumping fluid, valves used for regulating flow, actuators used for moving or controlling the micropump and valves and/or sensors used for sensing pressure and/or flow. The MEMS device incorporates one or more piezoelectric devices, as known to those skilled in the art. Examples piezoelectric devices include piezoelectric actuators and various types of MEMS sensors. As described in more detail below, the piezoelectric devices function as the active element(s) of a pump for pumping fluid, a valve for preventing fluid flow and/or a sensor for sensing pressure or flow. (However, various types of MEMS sensors can be used as the sensing elements of the architecture.) Further, other MEMS or non-MEMS structures or technology may also be used to achieve desired results as known to those skilled in the art.) Micropump 100 may be used in a drug infusion system for infusing a drug (i.e., medication) or other fluid to a patient (user). Medication may include small molecule pharmaceutical solutions, large molecule or protein drug solutions, saline solutions, blood or other fluids known to those skilled in the art. Insulin is an example fluid. However, micropump 100 may be used in other environments known to those skilled in the art.

Micropump 100 is configured to maximize micropump efficiency per mm² (i.e., stroke volume per unit area per Watt). To this end, micropump 100 is a cavity substrate that includes a chamber or cavity 102 that is configured to be any shape known to those skilled in the art to achieve desired results (i.e., pumping efficiency). Micropump 100 further includes (1) silicon on insulator (SOI) wafer 104 (top wafer) that incorporates a buried oxide (BOX) layer and silicon (Si) layer as known to those skilled in the art and (2) double sided polish (DSP) silicon wafer or layer 106. SOI wafer 104 sits between silicon wafer 106 and several piezoelectric actuators (transducers) 108, 110, 112 as shown and described below in more detail. A metallization and conductive epoxy layer 114 binds piezoelectric actuators 108, 110 and 112 to SOI wafer 104 as known to those skilled in the art. Wafer 106 includes inlet and outlet ports 121, 123 that communicate with chamber 102 via channels 117,119, respectively, that extend through the combined wafer structure (wafers 104,106) as shown. (Note that wafers 106 and wafer 130 may alternatively be SOI wafers as known to those skilled in the art.)

Micropump 100 further includes third wafer 130 (as described in more detail below) that incorporates inlet and outlet ports (openings) 116, 118 as well as channels 125, 127. Channels 125, 127 are configured to communicate with chamber 102 via channels 117, 119, respectively of the combined wafer structure (wafers 104,106). As shown, inlet and outlet ports 116, 118 are part of third wafer or layer 130 as shown (and described in more detail below.) Second wafer 106 and third wafer 130 define the structure of channels 125, 127, but it is the third wafer 130 that defines the structure of inlet and outlet ports 116, 118.

A silicon layer (within in SOI wafer 104 and silicon wafer 106) lines the surfaces of chamber 102 as well as channels 117, 119, 125, 127 throughout micropump 100.

(Silicon also lines inlet and outlet ports (openings) 116, 118 and channel segments within third layer 130 described in detail below). SOI wafer 104 functions as a membrane of micropump 100 that acts upon the fluid within chamber 102 (i.e., to pump fluid—displace or draw fluid into chamber 102 or to prevent fluid flow as a valve mechanism) in response to piezoelectric actuators 108, 110, 112. The action is deformation or deflection of layer 104 (membrane). Details appear below.

Micropump 100 includes a pump section 120 and two valve sections 122, 124 that function together to pump fluid through micropump 100. Pump section 120 includes chamber 122 and piezoelectric actuator 110 that is layered on top of silicon layer 104 and it functions to pump or deform/bend silicon layer 104 to draw into or displace liquid contents into chamber 102 as known to those skilled in the art. Valve sections 122 and 124 include inlet and outlet ports 121,123, respectively, and piezoelectric actuators 108 and 112 as well as valve seats 126, 128, respectively. Valve seats 126, 128 are configured to extend into chamber 102 and to define the introduction of channels 117, 119 and inlet/outlet ports 116, 118. As described above, piezoelectric actuators 108, 112 are layered on top of SOI wafer 104. piezoelectric actuators 108, 112 are configured to compress against SOI wafer 104 (membrane) to reach and seal valve seats 126,128 to thereby discontinue flow through inlet/outlets 116, 118, respectively as needed for proper pump performance, as known to those skilled in the art. (Note that a micropump may include any number of pumps and/or valves as described herein.)

In this example, micropump 100 incorporates a third silicon DSP or silicon on insulator (SOI) wafer or layer 130 that is bonded to (or otherwise joined to or integrated with) the bottom of the substrate (bonded to silicon wafer 106) for the purpose of integrating sensors 132, 134 in close proximity or adjacent to the inlet and outlet ports 116, 118. This close proximity of sensors 132, 134 include a location at the inlet and outlet ports 116, 118 as shown. As described above, third silicon layer 130 is configured with channel structure (125, 127) to communicate with and define inlet and outlet ports 116, 118. In this example, sensors 132,134 are embedded within cavities or apertures (as shown) along the bottom surface of silicon layer 130, in sections 130a, 130b in which layer 130 is narrower in width (also referred to as depth). Narrower sections 130a, 130b each function as a membrane configured to deflect in response to flow or pressure at inlet and outlet ports 118, 112 (and hence within the valve channels 117, 119 and chamber 102). That is, sensors 132, 134 are thus used to sense the deflection, i.e., pressure/flow at the inlet and outlet ports 118, 120. Sensors 132, 134 are preferably piezoelectric (material) for creating an electrical signal, causing sections (membranes) 130a and 130b to deflect. However, piezoelectric sensors are not the only mechanism or component that can be used as a sensor. Those skilled in the art know that other types of sensors with various sensing mechanisms may be used to sense pressure or fluid flow. Examples include capacitive sensors, piezoresistors (piezoresistive sensing) and MEMS flow sensors.

As stated above, sensing can be flow rate or pressure as known to those skilled in the art. By sensing at both the inlet and outlet ports, pressure differences within micropump 100 are obtained to derive valuable data of the performance of micropump 100. For example, by locating pressure sensors in proximity to the inlet and outlet ports, flow rate within the micropump may be calculated. In addition, abnormal function detection is also useful to detect dysfunction at different points in the fluid pathway in the actual insulin delivery device (system) in which the micropump is a component. Errors may be in the fluid pathway before reaching the micropump or after the micropump. In sum, the ability to sense within the micropump 100 and its fluidic pathway is a real benefit in gathering data to optimize performance of micropump 100. The information can also be used to optimize the key pumping parameters to be able to rectify flow at any given time. This is especially important when it comes to potential occlusions in the system as well as to monitor overall fluidic channel pressure. Further, sensing pressure in fluidic pathway can help avoid micropump failures and adverse events to the patient.

With the micropump configuration disclosed herein, abnormal function may be detected within the micropump itself or the delivery device in which the micropump is a component (along with a reservoir, MCU, tubing, needles, battery to name a few components).

With the micropump configuration disclosed herein, no additional wafers or other structures like an interposer are required to integrate MEMS sensors to sense flow or pressure in the fluidic channels in which the micropump is connected. As disclosed herein, sensors 132, 134 are piezoelectric sensors. However, any other type of sensor may be used to measure pressure/flow within the channels.

FIGS. 2-7 depict several stages and steps of the MEMS micropump fabrication along with its corresponding structure.

Specifically, as shown in FIG. 2, fabrication begins with an initial silicon substrate (also referred to as silicon microchip) with upper SOI wafer 104 and lower silicon wafer or layer 106 as bonded together. SOI wafer 104 comprises a thin oxide layer over a silicon layer as described above. Lower silicon wafer 106 has the chamber, valve and channel structures. There is additional silicon at each end to provide sufficient space for the fluidic channels.

In some detail, during fabrication, an SOI wafer 104 initially includes a buried oxide layer sandwiched between two silicon layers. The SOI wafer 104 is then bonded to silicon wafer 106. SOI wafer is then grinded and etched to remove the top layer of silicon in the SOI wafer. The buried oxide layer acts as an etch stop so that when etching is performed, the oxide layer remains. The resulting SOI wafer comprises an oxide layer and a silicon layer.

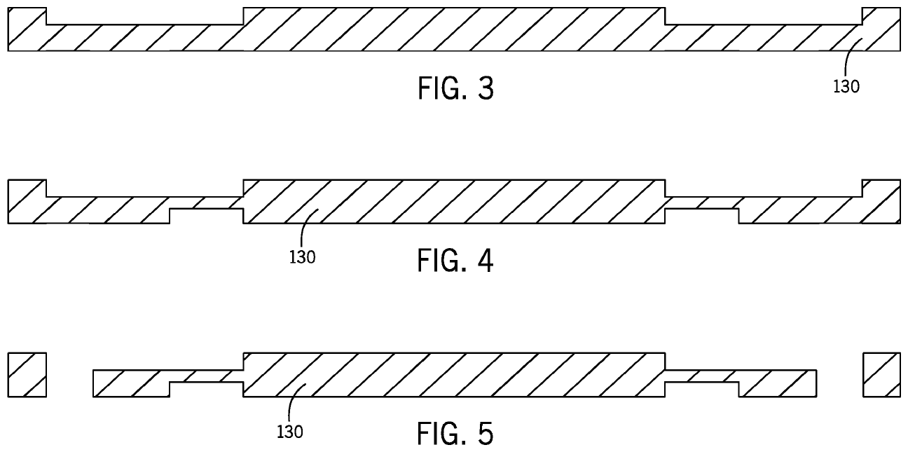

In FIG. 3, spincoat and photoresist patterning to achieve the desired structure design on the front side of the wafer is performed on third wafer or layer 130 of silicon. Then, dry etching is then performed on the top part of layer 130 to create channels in layer 130 as shown.

Then, as shown in FIG. 4, layer 130 is spincoat and pattern photoresist on the back side of the wafer is performed again on wafer 130 and dry etched to create membranes at the inlet and outlets.

Then, spincoat and pattern photoresist on the back side of the wafer is again performed on layer 130 as well as dry etching to complete and open up both the inlet and outlet channels. This is shown in FIG. 5.

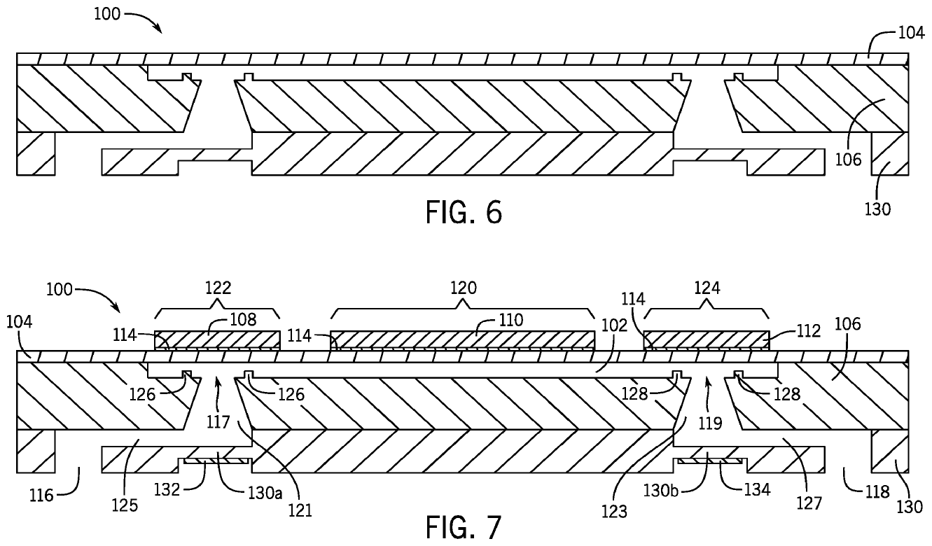

Next, as shown in FIG. 6, third layer 130 is then bonded to the current substrate (SOI wafer 104 and silicon wafer 106 in FIG. 2), making sure the channels are aligned as shown. Piezoelectric actuators and piezoelectric sensors are added as shown in FIG. 7.

It is to be understood that the disclosure teaches examples of the illustrative embodiments and that many variations of the invention can easily be devised by those skilled in the art after reading this disclosure and that the scope of the present invention is to be determined by the claims below.

1. A MEMS micropump comprising:
a pump section including a chamber that is configured to increase and decrease in volume as fluid is received and released from the chamber, respectively and an actuator for changing the volume within the chamber;
a first valve section including an inlet port communicating with the chamber to receive the fluid;
a second valve section including an outlet port communicating with the chamber to release the fluid from the chamber;
first and second wafers configured to define the chamber and enable it to communicate with the inlet port and outlet port and define a fluid pathway between the inlet port, chamber and outlet port,
wherein the first wafer is configured as a membrane that deforms in response to actuation of the actuator creating a pressure difference within the chamber as it increases and decreases in volume, thereby moving fluid into or out of the chamber via the inlet and outlet ports, respectively;
first sensor and second sensors, in proximity to the inlet and outlet ports respectively, for detecting abnormalities in fluid flow within the fluid pathway; and
a third wafer joined to the second wafer configured to integrate the first sensor and second sensor into the inlet port and the outlet port, respectively.

2. The MEMS micropump of claim 1 wherein the second and third wafers define channels that communicate with the chamber and the inlet port and the outlet port.

3. The MEMS micropump of claim 1 wherein the first and second sensors are configured to sense flow and/or pressure in proximity to the inlet and outlet ports.

4. The MEMS micropump of claim 1 wherein the first and second sensors are configured to sense pressure in proximity to the inlet and outlet ports to measure flow across the MEMS micropump.

5. The MEMS device of claim 1 wherein the third wafer includes first and second apertures in proximity to the inlet port or outlet port for receiving the first and second sensors.

6. The MEMS device of claim 1 wherein the third wafer includes first and second sections in proximity to the inlet and outlet ports respectively, each of the first and second sections function as a membrane that is configured to deflect in response to flow or pressure changes.

7. The MEMS micropump of claim 6 wherein the first and second sections are configured as a reduction in width as compared to a width of the third wafer.

8. The MEMS device of claim 2 wherein the actuator includes a piezoelectric device that actuates in response to an electrical signal causing the first wafer to deflect.

9. A MEMS device configured as a micropump with an inlet port to receive fluid and an outlet port to release the fluid from the micropump, the MEMS device comprising:
first and second wafers, the first wafer configured as a membrane;
a chamber defined by the first and second wafers for receiving fluid, the chamber configured to communicate with the inlet and outlet ports and defining a fluidic pathway between the inlet and outlet ports, wherein the first wafer is configured to deform creating a pressure difference within the chamber and thereby move fluid into or out of the chamber via inlet and outlet ports, respectively; and
first sensor and second sensors, in proximity to the inlet and outlet ports respectively, for sensing flow or pressure.

10. The MEMS device of claim 9 further comprising:
a third wafer bonded to the second wafer, wherein the second and third wafers define channels that communicate with the chamber and the inlet port and the outlet port and wherein the third wafer is configured to integrate the first sensor and the second sensor into the inlet port and outlet port respectively.

11. The MEMS device of claim 10 wherein the third wafer includes first and second apertures in proximity to the inlet port and outlet port for receiving the first and second sensors and for sensing flow and/or pressure at the inlet port or the outlet port.

12. The MEMS device of claim 10 wherein the third wafer layer includes first and second sections in proximity to the inlet and outlet ports respectively, each of the first and second sections that are reduced in width and functions as a membrane that is configured to deflect in response to flow or pressure changes.

13. The MEMS device of claim 10 wherein the first and second sensors include piezoelectric material or piezoresistors or capacitive sensors for creating an electrical signal with membrane deflection.
